(19) United States
(12) Patent Application Publication
(10) Pub. No.: US 2014/0277588 A1
(43) Pub. Date: Sep. 18, 2014

(54) SYSTEM AND METHOD FOR PROVIDING A PROSTHETIC DEVICE WITH NON-TACTILE SENSORY FEEDBACK

(71) Applicants: Eli Robert Patt, Sherman Oaks, CA (US); Joey Isaac Ben-Zvi, Los Angeles, CA (US); Alexander Robert Mosch, Los Angeles, CA (US)

(72) Inventors: Eli Robert Patt, Sherman Oaks, CA (US); Joey Isaac Ben-Zvi, Los Angeles, CA (US); Alexander Robert Mosch, Los Angeles, CA (US)

(21) Appl. No.: 14/217,053

(22) Filed: Mar. 17, 2014

Related U.S. Application Data
(60) Provisional application No. 61/800,741, filed on Mar. 15, 2013.

(51) Int. Cl. A61F 2/58

(52) U.S. Cl. CPC A61F 2/586; A61F 2002/5061
USPC 623/57

(57) ABSTRACT

A system and method for substituting vision based cues for lost sense of touch may employ a small electronic display or other non-tactile feedback mechanism that attaches to a prosthetic arm or other limb. Devices according to certain embodiments of the present invention include an array of pressure or other sensors that may be deployed against a prosthetic hand by structures such as finger cots or gloves containing the sensors. In one embodiment, an electronic display may be used to display a colored bar that moves from left to right, and changes colors along a gradient, as detected pressure on the sensors deposed against the hand increases. Movement across space and change in color are both easily discernable visual cues for these purposes.

### CROSS-REFERENCE TO RELATED APPLICATION

This application claims priority to and the benefit of U.S. Provisional Application No. 61/800,741, filed Mar. 1, 2013, the entire contents of which are incorporated herein by reference.

### FIELD OF THE INVENTION

The present invention is related to providing detection, processing, and feedback of sensory information to prosthesis users, and in particular, providing visual or other non-tactile sensory cues representing the tactile properties of an object being manipulated by a prosthetic device during gripping or other contact of objects.

### BACKGROUND OF THE INVENTION

There are over 1,900,000 people in the United States who use prosthetic devices. Each year 10,000 new upper extremity or arm amputations occur per year in the US.

In a 2008 study (1), 246 amputee patients were studied and of these only 126 were found to be frequent prosthesis users (i.e. those using their prosthetic arms two or more times per year). Of the frequent prosthesis users, 60% claimed that they are as, or more functional without the prosthetic arm. 53% of them agree that upper extremity prosthetic arms are too difficult to use for handling objects and are otherwise inconvenient to use. Forty-five percent say that they derive more sensory feedback while using other intact limbs over the prosthetic device.

The main reason for the lack of use of prosthetic arms is that standard prosthetic devices do not provide tactile feedback regarding the physical aspects of objects being handled by the prosthetic arm and fingers.

Efforts directed toward improved functionality of prostheses have taken two predominant developmental pathways, namely:

(1) highly evolved robotics hardware and software incorporating sensors, actuators, motors, filters, and algorithms in the form of prostheses to facilitate manipulation of objects by a user; and

(2) physiological interventions (ie. surgery) aimed at utilizing remaining portions of nerves from a displaced hand or arm to invoke motion in a prosthetic device, or to provide some sensory feedback.

However, these methods result in extremely expensive prostheses and often require surgery. The associated cost is prohibitive for the majority of prosthesis users and is often not covered by insurance. In addition the prostheses require extensive and costly surgery, which is also generally not reimbursable. Finally, the methods of the prior art require extensive training that generally involves rehabilitation experts, facilities, and equipment at further high cost.

An example of the robotics approach is an advanced prosthetic arm that recently became available commercially, which was developed through funding from DARPA (Defense Advanced Research Agencies). The “Luke arm” replicates 18 of the 22 degrees of motion of a normal human hand, the most currently available. With these capabilities the functionality for the user increases dramatically to allow more control in handling objects.

An example of the physiological approach in the advancement of prosthetic arms is the development of targeted nerve re-innervation, a procedure in which severed nerve endings from the hand and arm are moved and re-terminated on muscles in the pectoral region. An amputee attempts to control their prosthetic arm as if it were a real arm, stimulating the relocated nerves that now impact upon the pectoral muscles. The resulting movement of the pectoral muscles is then measured by sensors placed on the pectoral muscles, interpreted, and re-transmitted to the robotic hardware in the prosthetic arm, moving the arm.

In an attempt to incorporate sensory feedback into the Luke arm, a system that uses pressure-sensing tactors that vibrate in response to pressure was developed. These tactors are placed elsewhere on the body and the correlation between these vibrations and the sensory inputs they represent must be learned by the user. There are multiple issues with this approach using tactors, the first being that they attempt to replace the lost natural tactile sensations one would feel in the missing hand, arm, or other body part with alternate tactile responses induced at an alternate location on the body. Attempting to detect vibration in one location and interpreting it to explain other more complex tactile sensations due to complex interactions occurring at another location by a hand, arm, or other body part has been found to be extremely difficult for the prosthesis user. In fact many users attempting to learn this response mechanism complain that the resulting sensory input can be very “irritating” and that the consistent vibrations the devices produce are undesired and contain little or no real value.

Another method for recreating sensory feedback in the process of targeted nerve re-innervation evolved from the discovery that unexpected sensations occurred in the region of the chest to which the motor nerve endings were transferred. The patient interpreted these unexpected sensations as phantom sensations akin to the lost sensations in the missing hand (2). This led to new interventional procedures whereby sensory nerves were also transferred to the pectoral region along with the nerves responsible for movement. Following several developments in this area, patients were able to distinguish between different specific areas of their missing hands, including, at times, a distinction between different fingers (3). This ability to recreate lost sensation is potentially a great advancement for amputees, as it portends future methods of restoring the actual sensation of touch alongside movement. However as of this time, the science is still not there, and as an example of the complexity of incorporating re-innervation, studies have shown that when two locations of skin are simultaneously touched they integrate their senses and over time these inputs tend to blend into one unrecognizable sensation (4-6). A 2007 study on targeted nerve re-innervation showed that when sensory nerves from the hand are moved and re-terminated at new locations on the chest, a similar result occurs. When the skin in the area surrounding the transferred nerves is stimulated, the patient describes sensations being either in the missing hand or chest, at random, while mostly feeling an uncomfortable combination of the two (3). Re-directing sensory nerves from one location to an intact second location leads to unresolvable ambiguities in the brain (3-6).

Thus aside from the expense, there are also extreme complications in implementing robotics that induce vibrations on a different body part or that involve re-innervation to induce feeling on an alternate body part that must be interpreted to convey the feeling in the missing hand, arm or body part. The brain has difficulty interpreting such signals.

The robotics and physiological advancements described above are applicable to very few of those needing prostheses due their extremely high cost, the need in some cases for surgical intervention, and the lack of medical reimbursement for both the costly procedures and the costly prostheses. Thus, less costly and more widely applicable alternative approaches are needed to improve the lot of the majority of prosthesis users without requiring them to discard their existing prostheses. While the prior developments in the field are not intended for, nor can they be used as prosthetic “add-ons” or enhancement for a majority of prosthesis users, an easy to use and inexpensive “add-on” providing some improved functionality is needed and could deliver significant increase in satisfaction for amputees with the use of their prosthetic limbs.

### SUMMARY

A viable system has yet to be discovered for delivering sensory feedback from a prosthetic device in a manner conducive to improved handling of objects. Thus, it is desirable to provide a method for delivering sensory input from sensors deployed in the fingers or other portions of a prosthetic limb in such a manner as to provide sensory feedback in the form of readily identifiable and readily understandable cues.

Moreover, it is desirable that such a device could be added on to any pre-existing prosthetic limb, providing ease of use and convenience. This is important because prosthesis users could then keep their existing prosthetic devices, which they are already accustomed to, shortening the learning curve for adaptation of the added utility of the present invention.

The method of the invention takes advantage of the fact that immediately after fitting a prosthetic arm, in the absence of the lost sensory tactile input, the brain of an amputee naturally attempts to compensate with additional information derived from visualization of the objects being manipulated. In other words, it is natural when touch is lost, to try and incorporate additional visual cues to replace the lost tactile ones. This is found to be a more natural compensatory action than attempting to learn to replace lost touch with alternate tactile response in another part of the body.

As described above the brain naturally seeks to compensate for lost tactile sensation with additional visual cues to aid in interpretation of what would be felt by, for example, a missing limb. It might therefore be a more practical way to obviate some of the lost sense by providing readily accessible visual cues not just about the visible information at the interrogated site, but in fact by translating tactile information normally perceived by an intact hand or arm, including but not limited to pressure, texture, hardness, dryness, etc., into easily discernable and easily learnable visual cues, including but not limited to color, color variation, graphs, patterns, etc. that can be displayed on a readily visualized display worn for example on the limb, integrated into glasses or other headset, or otherwise conveniently placed so that it can be observed during a task. The visual cues may also include movement of a pattern across space or time and may incorporate changes of color or position on the display or other methods of displaying the data such that it is easier to interpret by a user while minimizing the extent of training it requires to be mastered.

Two visual cues that are easily recognized by the brain's visual pathways are movement across space and change in color. A display of a colored bar moving across a screen from left to right, and color changes along a gradient, are easily discernable cues.

With training and adaptation to use in performance of routine actions in daily life, a user's brain begins to train-itself itself to recognize color patterns that correspond to certain tasks leading to improvement in quality of life. Elimination of the need to purchase a new prosthetic, ease of use of the device described in the present invention, and simplicity of the learning profile for the device could potentially yield a high ratio of performance to cost, at the same time improving quality of life.

Embodiments of the present invention are designed to increase the satisfaction of prosthesis users with the functionality of their prosthetic hands, arms or other body parts by providing readily accessible visual feedback as to the objects they are attempting to manipulate with their prosthesis. Embodiments of the present invention can be in the form of new prosthetic devices incorporating the feedback methods, however embodiments of the present invention also include “add-ons” or “attachments” to commercially available prostheses.

In one embodiment of the invention, pressure sensors are placed against the fingers of the prosthetic hand. The pressure sensors may be encapsulated in a plastic material shaped like a finger cot. The finger cots are designed to fit snugly around the fingers of the prosthetic hand while still allowing for flexibility and mobility. The pressure sensors may each incorporate a single sensor element or a multiplicity of sensor elements. Complex forms of arrays of sensor elements may yield additional tactile information such as texture. Other types of sensors may be used to provide information such as temperature of the surface being touched.

The signals from the pressure sensors may be conditioned in a pre-amplifier and are conveyed to an electronic signal-processing unit (SPU). The SPU may contain electronic hardware, microprocessors, firmware and software that integrate and analyze the various sensor signals and otherwise process these signals using digital and analog electronics and computer programs implementing algorithms to interpret the physical interaction between the prosthesis and an object that the prosthesis manipulates. The interpreted signal is then further processed to produce an output signal that is conveyed to a feedback generator to create a non-tactile sensory feedback representative of the sensor output signal. This feedback generator may be a visual display unit, such as a miniaturized electronic display screen that can, for example, be worn comfortably over a prosthetic arm in the wrist region utilizing a wristband. The electronic display screen may be any known form of electronic display, including without limitation an LCD, LED, OLED, plasma, or other known display, or it may be one or more light sources driven in such a manner as to provide non-tactile feedback to the prosthesis user.

The output signal can be derived using various algorithms such that the LCD screen or other display device displays the information graphically. In one embodiment, the information is displayed as a pattern with a colored bar that moves from left to right or up or down, and/or changes colors along a gradient, as the detected pressure on the fingertip sensors increases. In another embodiment of the invention, as the sensed pressure increases the displayed colors may change over the visible light spectrum. Such a visual cue is easily discernable by the user and the correlation of such cues with the level of pressure being applied to the object is easily learnable for a wide variety of objects with practice. Moreover such practice can easily be performed in one's home and does not require expensive training at a medical or technology facility.

A system and method for providing non-tactile sensory cues representing the tactile properties of an object manipulated by a prosthetic device of a user in accordance with the invention includes at least one sensor mountable to the prosthetic device, the at least one sensor configured to detect tactile information of an object contacted by the prosthetic device and create a sensor output representing the detected tactile information; processing circuitry configured to receive the sensor output and create a non-tactile output corresponding to the sensor output; and a non-tactile feedback generator configured to generate a non-tactile sensory feedback in response to the non-tactile output of the processing circuitry for perception by the user.

In one embodiment, the non-tactile feedback generator provides visual feedback representing the detected tactile information, and may be an electronic display. The non-tactile feedback generator may use color to signify the tactile information, such as to signify the magnitude of the tactile information. The non-tactile feedback generator may also use at least one change in color, at least one shape, or at graphical representation to signify the tactile information.

In another embodiment, the non-tactile feedback generator may use light intensity to signify the tactile information, a time varying display, or numerals to signify the tactile information. The at least one sensor may communicate the sensor output to the processing circuitry wirelessly, and/or the processing circuitry may communicate the non-tactile output to the non-tactile feedback generator wirelessly, even to remote devices. The processing circuitry may also comprise a memory to store the tactile information, and may be configured to upload information for comparison with the tactile information. Alternatively, the non-tactile feedback generator may provide auditory feedback representing the detected tactile information.

In a further embodiment, the tactile information may comprise at least one property selected from the group consisting of texture, temperature, hardness, softness and phase of the object. The at least one sensor may comprise an array of similar sensors, or a plurality of sets of sensors, the sensors of at least two of the sets being of different types. Also, the at least one sensor may be affixed to a device worn on the prosthetic device and selected from the group consisting of a glove, a finger cot, a sock, a toe cot, a brace, or a cover fitted over at least a portion of the prosthetic device.

### BRIEF DESCRIPTION OF THE DRAWINGS

The above and other features of the present invention may be more fully understood from the following detailed description, taken together with the accompanying drawings, briefly described below, wherein similar reference characters refer to similar elements throughout and in which:

FIG. 1 shows a schematic side view of one configuration of a prosthesis, where a prosthetic hand is fitted with finger cots incorporating pressure sensors and a cuff display device for displaying visual cues (i.e., visual non-tactile feedback) related to the inputs on the pressure sensors according to an embodiment of the invention.

FIG. 2 shows a schematic front view of the configuration of FIG. 1 wherein the hand is rotated so that the palm is visible with the fingers open according to an embodiment of the present invention.

FIG. 3 shows a prosthesis where the prosthetic hand is fitted with a glove incorporating pressure sensors according to an embodiment of the present invention.

FIG. 4 is a partially fragmentary view of a glove according to FIG. 3 showing detailed aspects of the inside surfaces of the palmer and dorsal layers of the glove according to an embodiment of the present invention.

FIG. 5 is a detailed illustration of a cuff display device according to an embodiment of the present invention.

FIG. 6 is a schematic diagram of the major components of an embodiment of the present invention.

### DETAILED DESCRIPTION

In the following detailed description, only certain exemplary embodiments of the present invention are shown and described, by way of illustration. As those skilled in the art would recognize, the described exemplary embodiments may be modified in various ways, all without departing from the spirit or scope of the present invention. Accordingly, the drawings and descriptions are to be regarded as illustrative in nature, and not restrictive.

In one embodiment of the present invention shown in FIG. 1 and FIG. 2, tip pressure sensors 31 and pad pressure sensors 32 are placed against the fingers of the prosthetic hand. These pressure sensors may be encapsulated in a material such as a rubber or plastic conforming to the shape of the fingers to form finger cots such that they fit snugly around the fingers of the prosthetic hand while still allowing for flexibility and mobility. The pressure sensors 31 and 32 may be the same or different, and may incorporate a multiplicity of sensor elements 301-303 arranged in a circular, concentric, linear, or other form of sensor array. Complex forms of arrays of sensor elements may yield additional tactile information including but not limited to texture, hardness, softness, etc. Other types of sensors may be used to provide information about the nature or condition of the surface, such as its temperature, phase (i.e., whether it is liquid or solid), etc.

In another embodiment according to the present invention, a glove 40 that can be worn over a prosthetic hand 11 is provided as shown in FIGS. 3 and 4. The glove includes pressure sensors 33 along the fingers including at the finger tips 43 and in the palm 44 region of the glove. The pressure sensors are adhesively applied (i.e., glued) or otherwise affixed to the inside surface of the planter side of the glove as shown in FIG. 4. A preferred embodiment includes an arrangement deploying one or more of the pressure sensors in each articulating region of the prosthesis, however one skilled in the art will recognize that other arrangements whereby the sensors can be rearranged either permanently or at the time of use in order to optimize the input field of information to fit a particular type of prosthesis or task are evident.

The pressure sensors 33 may be the same throughout the arrangement on a glove or they may be different from each other, and may incorporate a multiplicity of sensor sub-elements arranged in a circular, concentric, linear, or other form of sensor array. Complex forms of sensor arrays may yield additional tactile information including but not limited to texture, hardness, softness, etc. Other types of sensors may be used to provide information about the nature or condition of the surface such as its temperature, whether it is liquid or solid, etc. The sensors 33 may be of any known type suitable for detecting pressure or other tactile information where the prosthetic hand or other limb contacts an object, and creating a sensor output signal representing the detected tactile information.

As illustrated in FIG. 6, the signals from the pressure sensors 33 may be conditioned in a pre-amplifier 101 and are conveyed by a plurality of wires, other conduit, or wireless channels 103 to an electronic signal-processing unit (SPU) 102. The SPU 102 contains electronic hardware, microprocessors, firmware and software that integrate and analyze the various sensor signals or otherwise process these signals using digital and/or analog electronics or computer programs implementing algorithms to interpret the physical interaction between the prosthesis and an object that the prosthesis manipulates. The interpreted signal is then further processed to produce a non-tactile output signal 104 that is conveyed to a visual display unit or other feedback generator 20 such as but not limited to a miniaturized LCD screen 21, for example, a 6 cm by 1.75 cm LCD screen. The display unit in one embodiment of the invention can be worn comfortably over the prosthesis in the wrist region of the prosthetic arm 11, as shown in FIG. 1 and FIG. 5, utilizing a wristband 50 that can be one of many alternatives. In one embodiment of the present invention the wristband is a simple band such as one used to attach a MP3 player to one's arm, such that it wraps around the forearm of the prosthesis and holds the screen tightly in place. The display unit 20 might alternatively be permanently mounted onto the prosthesis, free standing, or mounted on a fixture within the visual range of the user in other embodiments of the present invention. In still other embodiments of the present invention, the display unit 20 may be further miniaturized and embedded in eyeglass frames or other headset. In still other embodiments of the present invention the information may be conveyed to the user in the form of an auditory transmitter such that the audio signal varies in a way corresponding to the sensed inputs. The auditory transmitter may be a speaker, buzzer, or other device generating audible tone(s) signifying the tactile interaction between the prosthesis and the object being manipulated.

In an embodiment of the present invention the SPU 102 is housed inside the display unit 20 and connections 103 between the pressure sensors/preamplifiers and the SPU may be made individually or through a multi-element connector into the display unit 20, or wirelessly.

The unit may incorporate the ability of connecting any number of sensors of various types whereas the preferred embodiment has at least five sensor sleeves 30 to accommodate a full five-fingered prosthetic hand. Other embodiments are compatible with three-fingered prosthetic hands and still others accommodate gloves with a large number of sensors. In some embodiments part of the SPU is incorporated in the glove performing some of the sensor integration at the hand.

The output signal 104 can be derived using various algorithms such that the LCD screen or other display device, displays a pattern that may include but not be limited to a colored bar that moves from left to right or up or down, and may change colors in a manner including but not limited to along a gradient, as the detected pressure on the fingertip sensors increases. In one embodiment of the present invention the lowest pressure might be displayed using for example the color Violet illuminating the far left of the display. As the sensed pressure increases, as would happen for example as the user tightens their grip on an object, the color in one embodiment of the present invention might change with varying sensed pressure from Violet, to Indigo, to Blue, to Yellow, to Orange, to Red. When the colors change with varying pressure according to the visible-light spectrum, the meaning of the pattern and interpretation is easily understandable by people with very little training. In addition, the pattern might simultaneously move across the screen with varying pressure or other sensed input, so that for example, at the highest pressure a Red bar would be illuminated on the far right side of the screen. Such a visual cue as well as other visual cues would be easily discernable by the user. The correlation of such cues with the level of pressure being applied to the object would be easily learnable for a wide variety of objects with practice. Moreover such practice could be performed easily in one's home and would not require expensive training at a medical or technology facility as would be required for previously conceived robotic or surgical interventions used in prior improvements in prostheses.

The display unit (or “feedback generator”) in an embodiment of the present invention may also incorporate a second display 22 that can be used to convey other input data or messages to the user, including but not limited to a menu of user selectable inputs and tasks.

The display unit 20 in one embodiment of the invention illustrated in FIG. 5 has an input key 23 (FIG. 6) that can be used to select menu items provided by the user interface software. For example, as one starts to learn to use the device, one might select options via the input key or keys 23 to incorporate only pressure data into the algorithm and onto the display. Later as one becomes skilled with the level of input being learned for a task, one may select to incorporate additional features, more advanced algorithms, and more advanced display patterns that convey additional features about the object such as texture, temperature, etc.

In one embodiment of the invention a battery power system is deployed to provide power for the sensors, signal processing, and display. The battery may be of similar size and capacity to the batteries used in smart phones and may be interchangeable or rechargeable. The battery may have sufficient capacity to operate up to 24 hours between charges while delivering visual feedback in accordance with the invention. In one embodiment, power saving algorithms are incorporated into the display unit such that the quiescent pressure is measured and a threshold of input magnitude or rate of change of input magnitude or other measure of the need to turn on the power consuming components of the device such as the electronic display screen, which is otherwise in an “off” position and not consuming power from the battery.

In the context of the invention, various configurations of the sensors, encapsulating methods, algorithms, and display mechanisms whose configurations are readily evident to anyone skilled in the art may be used to accommodate the specific needs of various prostheses available on the market, including but not limited to hands, arms, feet, etc.

In this manner an amputee who already owns a prosthesis can attach a device of the present invention to the prosthesis to increase its utility. Thus, the described embodiments of the invention are usable with virtually any available prosthetic hand or foot, thereby accommodating many amputees. Additionally many embodiments of the invention permit the user to remove the device easily and at any time.

Using a prosthesis that a user is accustomed to and simply adding a device of the present invention enables a user to learn which motions and actions correspond to which display patterns and colors (indicating force, pressure, etc.). As users perform common tasks in their everyday routine, they become familiar with the specific display patterns and colors that communicate such information related to their actions. The user learns to recognize the surrogate visual cues offered by the invention in lieu of touch.

What is claimed is:

1. A system for providing non-tactile sensory cues representing the tactile properties of an object manipulated by a prosthetic device of a user, comprising:
at least one sensor mountable to the prosthetic device, the at least one sensor configured to detect tactile information of an object contacted by the prosthetic device and create a sensor output representing the detected tactile information;
processing circuitry configured to receive the sensor output and create a non-tactile output corresponding to the sensor output;
a non-tactile feedback generator configured to generate a non-tactile sensory feedback in response to the non-tactile output of the processing circuitry for perception by the user.

2. The system of claim 1 wherein:
the non-tactile feedback generator provides visual feedback representing the detected tactile information.

3. The system of claim 2 wherein:
the non-tactile feedback generator is an electronic display.

4. The system of claim 2 wherein
the non-tactile feedback generator uses color to signify the tactile information.

5. The system of claim 4 wherein:
the non-tactile feedback generator uses color to signify the magnitude of the tactile information.

6. The system of claim 4 wherein:
the non-tactile feedback generator uses at least one change in color to signify the tactile information.

7. The system of claim 2 wherein:
the non-tactile feedback generator uses at least one shape to signify the tactile information.

8. The system of claim 7 wherein:
the at least one shape comprises a graphical representation of the tactile information.

9. The system of claim 2 wherein:
the non-tactile feedback generator uses light intensity to signify the tactile information.

10. The system of claim 2 wherein:
the non-tactile feedback generator uses a time varying display to signify the tactile information.

11. The system of claim 2 wherein:
the non-tactile feedback generator uses numerals to signify the tactile information.

12. The system of claim 2 wherein:
the at least one sensor communicates the sensor output to the processing circuitry wirelessly.

13. The system of claim 2 wherein:
the processing circuitry communicates the non-tactile output to the non-tactile feedback generator wirelessly.

14. The system of claim 2 wherein:
the processing circuitry communicates the non-tactile output to remote devices wirelessy.

15. The system of claim 2 wherein:
the processing circuitry comprises a memory to store the tactile information.

16. The system of claim 14 wherein:
the processing circuitry is configured to upload information for comparison with the tactile information.

17. The system of claim 1 wherein:
the non-tactile feedback generator provides auditory feedback representing the detected tactile information.

18. The system of claim 1 wherein:
the tactile information comprises at least one property selected from the group consisting of texture, temperature, hardness, softness and phase of the object.

19. The system of claim 1 wherein:
the at least one sensor comprises an array of similar sensors.

20. The system of claim 1 wherein:
the at least one sensor comprises a plurality of sets of sensors, the sensors of at least two of the sets being of different types.

21. The system of claim 1 wherein:
the at least one sensor is affixed to a device worn by the prosthetic device and selected from the group consisting of a glove, a finger cot, a sock, a toe cot, a brace, or a cover fitted over at least a portion of the prosthetic device.

22. A method for providing non-tactile sensory cues representing the tactile properties of an object manipulated by a prosthetic device of a user, comprising:
providing at least one sensor mounted to the prosthetic device, the at least one sensor configured to detect tactile information of an object contacted by the prosthetic device and create a sensor output representing the detected tactile information;
applying the senor output to processing circuitry configured to receive the sensor output and create a non-tactile output corresponding to the sensor output;
applying the non-tactile output of the processing circuitry to a non-tactile feedback generator configured to generate a non-tactile sensory feedback for perception by the user.

23. The method of claim 22 wherein:
the non-tactile sensory feedback is visual feedback.

24. The method of claim 23 wherein:
the non-tactile feedback generator is an electronic display.

25. The method of claim 22 wherein:
the non-tactile sensory feedback is auditory feedback.
